(19) United States
(12) Patent Application Publication
(10) Pub. No.: US 2004/0156853 A1
(43) Pub. Date: Aug. 12, 2004

(54) Antibodies for cancer protection

(76) Inventors: Sanjay Awasthi, Arlington, TX (US); Sharad S. Singhal, Arlington, TX (US)

Correspondence Address: Monique A. Vander Molen, Gardere Wynne Sewell LLP, 3000 Thanksgiving Tower, 1601 Elm Street, Suite 3000, Dallas, TX, 75201-4767, US

(21) Appl. No.: 10/714,506

(22) Filed: Nov. 13, 2003

Related U.S. Application Data
(60) Provisional application No. 60/425,917, filed on Nov. 13, 2002.
(60) Provisional application No. 60/425,814, filed on Nov. 13, 2002.

(51) Int. Cl. A61K 39/395

(52) U.S. Cl. 424/155.1

(57) ABSTRACT

Generally, and in one form, the present invention is a method of treating one or more cancerous growing cells comprising the step of contacting one or more cancerous growing cells with an antibody to RLIP76, or a fragment of RLIP76. In yet another form, the present invention is a method of treating one or more cancerous growing cells comprising the step of contacting one or more cancerous growing cells with RLIP76 IgG. In still another form, the present invention is a pharmaceutical composition used to increases the accumulation of one or more chemotherapeutic agents inside a cancerous growing cell comprising one or more chemotherapeutic agents used in combination with an antibody to RLIP76, wherein the antibody comprises all or an effective portion thereof and when to bound a cancerous growing cell will promote the accumulation of one or more chemotherapeutic agents within the cell.

### CROSS-REFERENCES TO RELATED APPLICATIONS

This application claims the benefit of U.S. Provisional Patent Application No. 60/425,917, filed Nov. 13, 2002. This application herein incorporates by reference U.S. Provisional Patent Application Serial No. 60/425,814, filed on Nov. 13, 2002.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms CA 77495 awarded by the National Institutes of Health.

### BACKGROUND OF THE INVENTION

The present invention relates to the treatment of cells, and more specifically to a treatment for cells undergoing uncontrolled cell growth.

Cancer encompasses a larger group of related diseases that begin in cells that continue to divide. The uncontrolled growth of such cells generally results in the formation of a mass of tissue or tumor that can be malignant, many of which are metastatic, or benign. Aggressive cancers are generally those that undergo metastasis and have proven harder to cure, such as cancers of the lung, bone, and non-Hodgkins lymphomas. With many of the more aggressive cancers, largely fatal malignancies are often diagnosed, where the presence of locally advanced disease at the time of diagnosis is common, and precludes potentially curative surgical therapy in the majority of patients.

Metastatic disease, when found at the time of diagnosis, is often located in several parts of the body including the brain, bone/bone marrow, liver, adrenal gland or contralateral lung, and/or spleen and precludes not only surgical therapy, but also renders radiation therapy as merely a palliative modality. For example, with lung cancer, chemotherapy combined with chest irradiation is the cornerstone of attempted curative intent therapy in patients with limited stage small cell lung cancer (SCLC) as well as in locally advanced non-small cell lung cancer (NSCLC). In patients with metastatic SCLC or NSCLC, chemotherapy along with local irradiation to selected metastatic sites is frequently employed, largely as palliative therapy. The outcome of patients treated with combined chemotherapy and radiation in locally advanced disease is quite poor.

The underlying causes of failure of therapy in advanced disease stages are multiple, and include the inability to deliver effective doses of chemotherapy or irradiation due to unacceptable toxicity in patients who are generally in poor physical condition and have numerous co-morbid conditions. Inherent or acquired alterations in cellular metabolism or signal-transduction pathways governing cell growth, metastases, and apoptosis are suggested as major mechanisms contributing to the failure of therapy. Drug-accumulation defects present in NSCLC cells, for example, have been identified as significant contributors to the drug-resistant phenotype of these cells.

Primary active efflux mechanisms for chemotherapeutic agents are major contributors to the drug-accumulation defective phenotype. Similar mechanisms occur with regard to radiation therapy, where the accumulation of radiation by-products is overridden in such cancerous cells and results in a radiation resistant phenotype. Cancerous or continually growing cells exhibiting radiation- and drug-resistant phenotypes can occur in either malignant or benign growths, but are more pervasive in malignant growths, especially those that become metastatic.

The ATP-binding cassette (ABC)-family of drug-efflux pumps, particularly P-glycoprotein (Pgp, MDR-1 gene product) and multidrug-resistance associated proteins (MRPs) were originally thought the prime proteins involved in the drug-accumulation phenotype of cells (e.g., drug and multi-drug resistance). The activities of these proteins now appear insufficient to completely account for the drug-accumulation defects seen in drug-resistance. Clearly, then, there remains a need in identifying additional contributor(s) to the drug resistance phenomenon that occurs in the majority of patients who receive any type of prolonged chemotherapy or radiation therapy.

### SUMMARY OF THE INVENTION

The present invention solves the current problem associated ineffective treatments for cells undergoing uncontrolled growth. The present invention identifies the function of a protein, RLIP76, a novel non-ABC-family multi-specific transporter, as being largely responsible for the chemotherapeutic-resistant and radiation-resistant phenotype of continually growing cells, because of its significant overall contribution to chemotherapy- and radiation- by-product efflux. As presented herein, RLIP76 plays a significant role in the treatment of both aggressive and non-aggressive growing cells (e.g., cancerous growths). The protein is a specific target to overcome drug-resistance and drug-accumulation defective phenotypes in such growing cells. The present invention discloses that RLIP76 significantly enhances the cytotoxicity of chemotherapeutic agents and radiation therapy in cells undergoing uncontrolled growth.

Using specific antibodies that recognize RLIP76 on the cell surface, the antibodies are found to block the function of RLIP76. Furthermore, the highly specific antibodies enhance the cytotoxicity of chemotherapeutic agents in cancerous growing cells (e.g., SCLC and NSCLC). These results confirm the specificity of antibodies generated against RLIP76 (e.g., anti-RLIP76 IgG) and demonstrate the ability of the antibodies to specifically recognize RLIP76 on the surface of intact growing cells. The antibodies also induced apoptosis in such cells undergoing uncontrolled growth.

Generally, and in one form, the present invention is a method of treating one or more cells undergoing uncontrolled growth comprising the step of contacting one or more cells with an antibody to RLIP76. Cells may be any cell undergoing uncontrolled growth including cancerous cells from the lung, NCI-H82, NCI-H182, NCI-1417, NCI-1618, NCI-H1395, NCI-H2347, HCC44 (adenocarcinoma), and NCI-H2126 (large cell), and combinations thereof. As used here, the antibody to RLIP76 inhibits the transport activity of RLIP76 and may be used in combination with a drug, chemotherapy or radiation therapy to enhance the effectiveness of the drug or therapy. The antibody is selected from the group consisting of a mononclonal antibody, a polyclonal antibody, RLIP76 IgG raised against an effective portion of RLIP76.

In yet another form, the present invention is a pharmaceutical composition for the treatment of one or more cells undergoing uncontrolled growth comprising an antibody to RLIP76, wherein the antibody comprises all or an effective portion thereof that effectively reduces the transport activity of RLIP76 and a pharmaceutically effective carrier. Uses of the antibody are similar to that described above in addition to promoting cellular apoptosis.

The present invention is also a method of locating a cell undergoing uncontrolled growth comprising the step of contacting one or more cells with an antibody to RLIP76, wherein the antibody is connected to tracer molecule and the tracer molecule is capable of identifying the location of the cell. The antibody is administered to mammal using methods known to one of ordinary skill, including by injection, orally, dermally, and by infusion.

The present invention shows that RLIP76 is a therapeutic target for developing anti-neoplastic therapies for the treatment of cells undergoing uncontrolled growth. Antibodies raised against RLIP76 serve as highly effective therapies to be used alone or in combination with chemotherapeutic drugs or with radiation. The antibodies may be administered in a number of ways, depending on the location of the cancerous growth. Because the anti-RLIP76 antibodies may be considered for monotherapy as well as combination therapy, they may be considered as an effective alternative to chemo- or radiation therapy in order to treat or eradicate such growing cells. In all embodiments of the present invention, the antibody to RLIP76, or a fragment of RLIP76, alone or in combination may at any time be administered to a mammal in need thereof. Routes of administration are any of those available for compositions comprising an antibody and are readily apparent to those of skill in the art of preparing pharmaceutical compositions.

Those skilled in the art will further appreciate the above-noted features and advantages of the invention together with other important aspects thereof upon reading the detailed description that follows in conjunction with the drawings.

### BRIEF DESCRIPTION OF THE DRAWINGS

For more complete understanding of the features and advantages of the present invention, reference is now made to the detailed description of the invention along with the accompanying figures, wherein:

FIG. 1 depicts the DOX transport in artificial liposomes reconstituted with purified RLIP76 from SCLC and NSCLC cell lines;

FIG. 2 depicts the comparison of DOX and VCR transport rates in crude membrane vesicles prepared from SCLC and NSCLC cell lines, wherein ATP-dependent uptake of [14]-¹⁴C-DOX (panel A) and ³H-VCR (panel B) were determined in crude membrane vesicles from each of 6 SCLC and 6 NSCLC cell lines and each value represents mean of triplicate determinations with error bars representing standard deviations;

FIG. 3 depicts the dose-dependent inhibition of DOX transport in crude membrane vesicles from SCLC and NSCLC cells by antibodies to RLIP76, MRP and Pgp, wherein, the ATP-dependent DOX uptake rate was determined in crude membrane vesicles prepared as described from the H182 (∘) and H1618 (□) SCLC and H226 (⋄) and H2347 (Δ) NSCLC cell lines and as a control, purified recombinant RLIP76 (X) reconstituted in artificial soybean asolectin;

FIG. 4 depicts the relative contribution of RLIP76 to total DOX transport in crude membrane vesicles from SCLC;

FIG. 5 depicts the contribution of RLIP76 to total DOX transport in crude membrane vesicles from NSCLC;

FIG. 6 shows the membrane association of RLIP76 in lung cancer cell, with SDS-PAGE (top panel) and Western-blot against anti-RLIP76 IgG (middle-panel), and Western-blot against RLIP76-antigen-affinity column eluate of anti-RLIP76 IgG (bottom panel);

FIG. 7 depicts cell-surface localization of RLIP76 in lung cancer cell, with two SCLC (H82 and H1618) and two NSCLC (H520 and H2126) analyzed by flow-cytometry for cell surface expression of RLIP76 (panel A). Percent of positive cells in the absence of stain (first bar), secondary antibody alone (second bar), pre-immune IgG and secondary antibody (third bar), and anti-RLIP76 IgG and secondary antibody (fourth bar). Immunohistochemical localization of RLIP76 in H358 (panel B) was carried out with cells grown on a cover slip and treated with 4% paraformaldehyde fixative or with PBS alone (unfixed). Primary antibody used was pre-immune IgG or anti-RLIP IgG without absorption against an RLIP76 antigen column (top four panels) or after RLIP76 antigen column absorption (bottom two panels);

FIG. 8 depicts dose dependence of anti-RLIP76 IgG on IC₅₀ of DOX The IC₅₀ of DOX was determined for two SCLC cell lines, H82 (∘) and H182 (⋄), and two NSCLC cell lines, H2126 (Δ) and H2347 (□) in the presence of varying concentration of pre-immune IgG (open symbols) or anti-RLIP76 IgG (closed symbols). Each point represents the mean of 8 replicates in 3 separate experiments (n=24);

FIG. 9 depicts the effect of anti-RLIP76 IgG on DOX cytotoxicity, wherein survival-fraction was determined from MTT assay performed as previously described with DOX concentrations ranging from 0-3 μM and plotted on a semi-log scale and the values are presented as mean±SD from three separate determinations with eight replicates each (n=24), such that SCLC (upper panel), NSCLC (lower panel) and square is no antibody; open circle is pre-immune IgG; filled circle is anti-RLIP76 IgG;

FIG. 10 depicts anti-RLIP76 IgG induced apoptosis in SCLC and NSCLC, wherein DNA laddering was visualized by 1% agarose gel electrophoresis and DNA is extracted from cells, such that anti-RLIP76 IgG or pre-immune IgG was at 50 μg/ml and the concentration of DOX used for SCLC cell lines, H82 and H1417, was 0.06 μM, and for NSCLC cell lines, H358 and H1395, was 0.7 μM as compared with Lane 1 that contained lambda-DNA\HIND-III fragments in all panels;

FIG. 11 depicts immunohistochemical localization of RLIP76, trastuzumab and rituximab on H226 and H2126;

FIG. 12 depicts the synergy between DOX, anti-RLIP76, trastuzumab and rituximab with NSCLC H2126, wherein the combinations used were: Panel A, DOX and trastuzumab; Panel B, DOX and anti-RLIP76; Panel C, DOX, anti-RLIP76 and trastuzumab; Panel D, DOX and human IgG; Panel E, DOX and rabbit IgG; and Panel F, DOX, human IgG and rabbit IgG;

FIG. 13 depicts the synergy between anti-RLIP76, trastuzumab and rituximab with NSCLC H2126, wherein the combinations used were: Panel A, Anti-RLIP76 and trastuzumab; Panel B, Anti-RLIP76 and human IgG; Panel C, Anti-RLIP76 and rituximab; Panel D, trastuzumab and rituximab; Panel E, trastuzumab and human IgG; Panel F, rituximab and human IgG;

FIG. 14 depicts apoptosis caused by anti-RLIP76 in NSCLC cell lines, wherein lane 1 contained λDNA HIND III and cellular DNA was applied to lanes 2-8 as follows: lane 2, untreated DNA; lane 3, trastuzumab treated; lane 4, rabbit pre-immune IgG treated; lane 5, anti-RLIP76 IgG; lane 6 anti-RLIP76 and trastuzumab; lane 7, human IgG; and lane 8, rituximab;

FIG. 15 depicts the effect of anti-RLIP76 on 4HNE accumulation in lung cancer cells; and

FIG. 16 shows the effects of antibodies on efflux of DOX from intact NSCLC cells with NSCLC cell lines H226 (top panel) and H2126 (bottom panel).

### DETAILED DESCRIPTION OF THE INVENTION

Although making and using various embodiments of the present invention are discussed in detail below, it should be appreciated that the present invention provides many inventive concepts that may be embodied in a wide variety of contexts. The specific aspects and embodiments discussed herein are merely illustrative of ways to make and use the invention, and do not limit the scope of the invention.

In the description which follows like parts may be marked throughout the specification and drawing with the same reference numerals, respectively. The drawing figures are not necessarily to scale and certain features may be shown exaggerated in scale or in somewhat generalized or schematic form in the interest of clarity and conciseness.

The following are abbreviations that may be used in describing the present invention: RLIP, ra1 interacting protein; SCLC, small cell lung cancer; NSCLC, non-small cell lung cancer; GAP, GTPase activating protein; DOX, doxorubicin; MRP, multi-drug resistance associated protein; MDR, multi-drug resistance; GS-E, glutathioneelectrophile conjugates; VCR, vincristine.

As used herein, a “proteoliposome” is generally a protein and lectin or glyco- or phospholipid combination that forms a spherical micellular-like or vesicular structure. The structures may form spontaneously or by chemical or mechanical manipulation, or combinations thereof. Proteoliposomes take advantage of the amphipathic nature of the lipid (or lectin) that causes them to form bilayers when in solution resulting in at least one of several shapes, including: (a) spherical micelle with the tails inward, or (b) bimolecular sheets that are bilayers with hydrophobic tails sandwiched between hydrophilic head groups. In general proteoliposomes may reseal themselves when torn or broken. Proteoliposomes may contain only one lectin or lipid or a variety and combination of each. Examples of phospholipids include phosphatidylcholine, sphingomyelin, phosphatidylserine, inositol phospholipids, and phosphatidylethanolamine. When used, proteoliposomes may be charged or electrically neutral and are generally used at physiological pH. They may also be structures mixed with detergent (e.g., detergent/lipid/protein, detergent/lectin/protein). Methods for preparing proteoliposomes of defined lipid-protein or lectin-protein ratios and size are well-known to one of ordinary skill in the art of molecular biology and protein/lipid biochemistry.

“Toxic compounds” as used herein may xenobiotics, radiation, toxins, waste products, by-products of larger organic or inorganic molecules and/or may result from damage to such molecules. Stress is one example of damage. Other damages may be environmentally-induced, metabolically-induced, drug-induced, chemically-induced, radiation-induced, and physiologically induced, as examples. The toxic compounds may be in a mammal or occur in the environment or come from manufacturing and/or chemical processes that produce waste products. Toxic compounds, “toxic organic chemicals,” and “xenobiotics” are often used interchangeably. Toxic compounds may also include crude oil, crude oil fraction, an organic or inorganic chemical compound, radiation, a chemical solvent, metabolite, metabolic by-product, a chemical warfare agent, drug, drug by-product, chemical by-product, and combinations thereof.

As used herein, “uncontrolled cell growth,” “cancerous growing cell,” or “cancer cell” may be used to identify a cell that has lost control of normal growth control (including hyperplasia and dyplasia). In the organ, gland, or tissue of interest, the rates of new cell growth and old cell death are no longer in balance, cell growth is uncontrolled or there is loss in the ability of one or more cells to undergo “apoptosis.” The increase in the number of dividing cells may be rapid or gradual creating a growing mass of tissue called a “tumor” or “neoplasm.” Cancer growing cells and the tissue they comprise have a distinctive appearance (e.g., as viewed under the microscope), including a large number of dividing cells, variation in nuclear size and shape, variation in cell size and shape, loss of specialized cell features, loss of normal tissue organization, and a poorly defined tumor boundary. Apoptosis, or “cell suicide,” is the mechanism by which old or damaged cells normally self-destruct or die, where sometimes the death may be considered premature.

The term “protein,” as used herein, is meant to include any chain of amino acids and includes peptides, polypeptides, proteins, recombinant proteins, and modified proteins, such as glycoproteins, lipoproteins, phosphoproteins, metalloproteins, and the like.

Antibodies generated against RLIP76 may be monoclonal or polyclonal. As used herein, an “antibody” is an immunoglobulin, a solution of identical or heterogeneous immunoglobulins, or a mixture of immunoglobulin. A “monoclonal antibody” is an antibody expressed by one clonal cell line. As used herein, the term generally refers to a population of antibody molecules that contains only one species of an antigen binding site capable of immunoreacting with a particular epitope of a particular antigen. A “polyclonal antibody” is a mixture of heterogeneous antibodies. Typically, a polyclonal antibody will include myriad different antibodies molecules that bind a particular antigen or particular organism with at least some of the different antibodies immunoreacting with a different epitope of the antigen or organism. As used herein, a polyclonal antibody can be a mixture of two or more monoclonal antibodies.

An “antigen-binding portion” of an antibody is contained within the variable region of the Fab portion of an antibody and is the portion of the antibody that confers antigen specificity to the antibody (i.e., typically the three-dimensional pocket formed by the complementarity-determining regions of the heavy and light chains of the antibody). An “Fab portion” or “Fab region” is the proteolytic fragment of a digested immunoglobulin that contains the antigen-binding portion of that immunoglobulin. A “non-Fab portion” is that portion of an antibody not within the Fab portion, e.g., an Fc portion.

A “constant region” of an antibody is that portion of the antibody outside of the variable region. Generally encompassed within the constant region is the “effector portion” of an antibody, which is the portion of an antibody that is responsible for binding other immune system components that facilitate the immune response. Thus, for example, the site on an antibody that binds complement components or Fc receptors (not via its antigen-binding portion) is an effector portion of that antibody. Also included within the constant region is the “Fc region” or “Fc portion” is the proteolytic fragment of a digested immunoglobulin that does not contain the antigen-binding portion of that immunoglobulin.

When referring to a protein molecule such as an antibody, “purified” means separated from components that naturally accompany such molecules and relatively free from the proteins or other naturally-occurring organic molecules with which it is naturally associated. Purity can be measured by any appropriate method, e.g., column chromatography, polyacrylamide gel electrophoresis, or HPLC analysis. A chemically-synthesized protein or other recombinant protein produced in a cell type other than the cell type in which it naturally occurs is “purified.” An antibody containing a desired immunoglobulin type and an undesired immunoglobulin type is “enriched” for the desired immunoglobulin type when treatment of the antibody results in a higher ratio of desired immunoglobulin to undesired immunoglobulin after treatment then before treatment. For example, a solution of antibody containing Protein A-binding immunoglobulins and non-Protein A-binding immunoglobulins is enriched for the latter when some of all of the Protein A-binding antibodies are removed from the solution.

As used herein, “bind,” “binds,” or “reacts with” is meant that one molecule recognizes and contacts a particular second molecule in a sample, but does not substantially recognize or contact other molecules in the sample (e.g., Protein A “binds” to the constant region of Human IgG1 but not to Chicken IgG). Generally, an antibody that “specifically binds” another molecule has a particular binding affinity for the other molecule that exceeds that of random contacts and attachments that may occur in solution.

A “therapeutically effective amount” or “pharmaceutically effective amount” is an amount capable of producing a medically desirable effect in a treated animal or human (e.g., improvement in a disease symptom, reduction in uncontrolled growth). As used herein, “an effective portion of RLIP76,” is any combination of proteolytic peptide products of RLIP76 that, when combined, promotes the transport or prevents the accumulation of toxic organic compounds and/or enhances resistance to the toxic compounds. The effective portion may be a recombinant RLIP76.

Any conventional eukaryotic or bacterial expression vectors, of which many are known in the art, may be used in the practice of this invention to transfect mammalian cells or bacterial cells with the claimed proteoliposome. “Transfection” as used herein, may refer to the incorporation of a nucleic acid or protein into a cell by any means readily known in the art of molecular biology. As examples, transfection may include incorporation by proteoliposomes, electroporation, by viral incorporation, or by a nucleic acid-containing structures (e.g., expression vector or plasmid) and combinations thereof. The eukaryotic cell expression vectors described herein may be synthesized by techniques well known to those skilled in this art. The components of the vectors such as the bacterial replicons, selection genes, enhancers, promoters, and the like may be obtained from natural sources or synthesized by known procedures. Expression vectors useful in practicing this invention may also contain inducible promoters or comprise inducible expression systems as are well known in the art. The expression vectors may be introduced into the host cells by purely conventional methods, of which several are known in the art.

All technical and scientific terms used herein have the same meaning as commonly understood by one of ordinary skill in the art to which this invention belongs, unless defined otherwise. All publications mentioned herein are incorporated herein by reference to disclose and describe the methods and/or materials in connection with which the publications are cited. A further description of the present invention is herein provided.

Many chemotherapeutic agents are effective and widely used as antineoplastic agents. Some examples of these agents include fentanyl citrate, dolasetron mesylate, pamidronate, anastrozole, exemestane, bleomycin, busulfan, alemtuzumab, irinotecan, celecoxib, daunorubicin, cytarabine, doxorubicin (DOX), cabergoline, clonidine, amifostine, methotrexate, etoposide phosphate, toremifene, letrozole, gemcitabine, imatinib mesylate, carmustine, trastuzumab, topotecan, interferon alfa, granisetron, leuprolide, gemtuzumab, filgrastim, oprelvekin, nilutamide, tamoxifen, mitoxantrone, denileukin, diftitox, alitretinoin, BCG, porfimer, aldesleukin, samarium, rituximab, octreotide acetate, paclitaxel bexarotene, docetaxel, temozolomide, triptorelin, arsenic trioxide, methoxsalen, valrubicin,, leuprolide, capecitabine, ondansetron, goserelin, zoledronic acid, and allopurinol, and are administered by differing routes including injection, infusion, liposomal and/or implant, as examples.

As is often found, many treatments are effective for one form of cancer but not another. For example, DOX is effective for the treatment of SCLC, but has shown disappointing results in treatment of NSCLC. Biochemical mechanisms of differential response of NSCLC and SCLC to DOX are complex and incompletely understood. One DOX-resistance mechanism (inherent and acquired) in cancerous growing cells is from drug-accumulation defects related to multi-specific drug-transport proteins including the MDR1 gene product, P-glycoprotein (Pgp) and multidrug resistance associated proteins (MRP). For example, MDR1 gene product, Pgp, has been detected in both SCLC and NSCLC. However, MDR1 gene expression or protein has not been found to correlate with chemosensitivity, clinical response to therapy, tumor progression, drug-resistance, histology, or stage of malignancy.

MRP-family proteins have also been detected in nearly all cancerous cell lines or tumor tissue. Overexpression of MRP1 in drug-selected or transfected cell lines is clearly correlated with DOX resistance. MRP expression in many types of tumor tissue samples correlates with drug-resistance and prognosis, but is not an independent prognostic factor predicting drug-resistance. For cells such as SCLC, as an example, there have been observed difference in inherent drug-sensitivity to anthracyclines and alkylating agents in SCLC as compared with NSCLC. These differences have not been explained satisfactorily by expression of one protein or family of proteins.

The present invention shows that RLIP76, a ras-family GTPase activating protein (GAP), functions as an ATP-dependent transporter of radiation, chemotherapeutic agents as well as GS-E in isolated artificial liposomes. Whether the transport activity of RLIP76 contributes to inherent or acquired drug-accumulation defects in cancerous growing cells is not known. The studies described herein show that RLIP76 is present on cancerous cell lines and tissues. In addition, there is a correlation between chemotherapy or radiation therapy resistance and the ATPase activity of RLIP76 (data not shown). In many cases there is a 1:1 stoichiometry between the ATPase activity of RLIP76 and its transport activity.

Experimental Methods

Examples of reagents. [¹⁴C]-DOX (specific activity 57 mCi/mmol) and [³H]-VCR sulfate (specific activity 6.2 Ci/mmol) were used. SM-2 Bio-Beads used for preparation of artificial liposomes were purchased from Bio-Rad Laboratories, Hercules, Calif. Reagents for SDS-PAGE and Western blotting, DOX, C-219 antibodies against Pgp (mdr-1 gene product) were commercially purchased. Polyclonal guinea pig-anti human MRP1 antibodies were kindly provided by Dr. Guido Zaman, Netherlands Cancer Institute, Amsterdam. DE-52 and protein-A purified pre- and post-immune rabbit anti-human RLIP76 IgG fractions used in these studies as well as DNP-SG and DNP-SGSepharose-4B affinity resin were prepared using standard methods known to one of ordinary skill. Aliquots of proteins and antibodies were stored at −86 degrees Centigrade.

Examples of cell lines and culturing. Human SCLC cell lines, NCI-H69, NCI-H82, NCI-H182, NCI-H378, NCI-H1417, NCI-H1618 and NSCLC cell lines NCI-H1395, NCI-H2347 (adenocarcinoma), NCI-H520, NCI-H226 (squamous cell carcinoma), NCIH-358 (Bronchioalveolar) and NCI-H2126 (large cell) cell lines were obtained. All cells were cultured at 37 degrees Centigrade in a humidified atmosphere of 5 % CO₂ in RPMI-1640 medium supplemented with 10% (v/v) heat-inactivated FBS, 1% (v/v) P/S solution, 2 mM L-glutamine, 10 mM HEPES, 1 mM sodium pyruvate, 4.5 g/L glucose, and 1.5 g/L sodium bicarbonate.

Examples of purification of RLIP76 from SCLC and NSCLC. DNP-SG-Sepharose affinity purification of RLIP76, as well as ATPase activity determinations to monitor transport, SDS-PAGE and Western blot analyses to analyzed purified protein, and protein determination by the methods readily apparent to those of ordinary skill in the art of molecular biology and biochemistry.

Examples of reconstitution of purifed RLIP76 into artificial liposomes. Functional reconstitution of RLIP76 purified from the H182, H1618 (SCLC), H226, and H2347 (NSCLC) cell lines used techniques readily apparent to those of ordinary skill in the art of molecular biology and biochemistry.

Examples of preparation of crude membrane vesicles. Crude membrane vesicle fraction was prepared from the human lung cancer cell lines using techniques readily apparent to those of ordinary skill in the art of molecular biology and biochemistry. Crude vesicles were enriched for the in-side-out vesicles by passing over a wheat germ agglutinin-Sepharose column (1 cm×3 cm), which selectively retains the right side out vesicles.

Examples of transport measurements. Transport studies of DOX and VCR in crude membrane vesicles and artificial liposomes were performed using techniques readily apparent to those of ordinary skill in the art. ATP-dependent uptake of 14-[¹⁴C]-DOX (specific activity 8.4×10⁴ cpm/nmol) was determined by subtracting the radioactivity (cpm) of the control without ATP from that of the experimental containing ATP, and the transport of DOX was calculated in terms of pmol/min/mg protein. The transport of [³H]-VCR (specific activity 5.8×10⁴ cpm/nmol) was measured similarly.

Examples of transport of DOX in vesicles coated with antibodies. Crude membrane vesicles (20 μg protein/30 μl reaction mixture) prepared from different human lung cancer cell lines were incubated separately with pre-immune IgG, anti-RLIP76 IgG, anti-MRP, anti-Pgp antibodies, or mixture of the antibodies for 30 min at room temperature. In one of the controls, IgG was excluded while the other control was treated with an equal amount of pre-immune IgG. After incubation, the ATP-dependent transport of 14-[¹⁴C]-DOX in IOV coated with different antibodies was measured as described previously.

Functional Reconstitution of Purified Cancer Cell RLIP76 in Artificial Liposomes

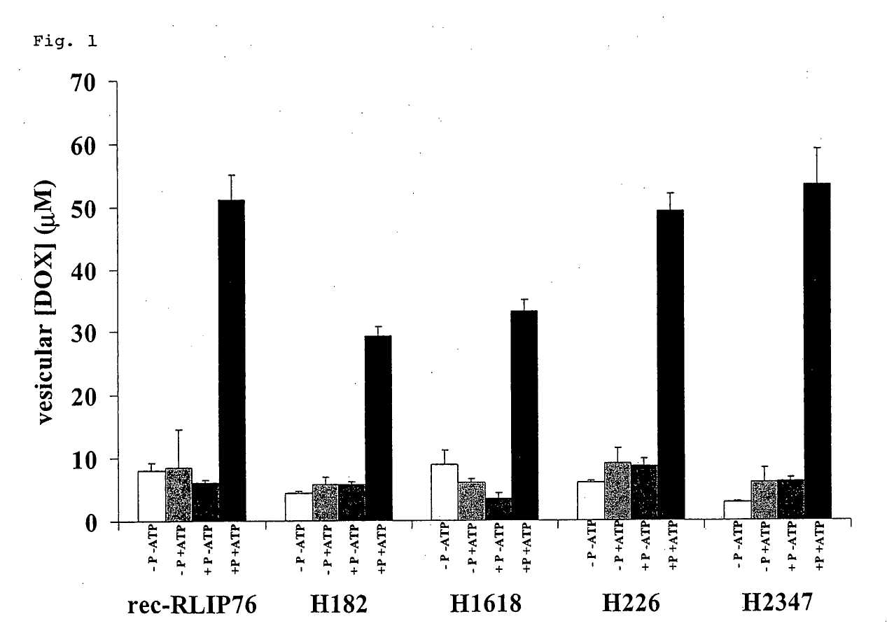

RLIP76 was purified from the H182 and H1618 SCLC cell lines, the H226 and H2347 NSCLC cell lines, and from E. coli expressing recombinant RLIP76 as previously described. [See Singhal, et al. (2001) Acta Biochim. Pol. 48, 551-562; herein incorporated by reference.] Purified protein fraction was subjected to SDS-PAGE and Western-blot, and amino acid composition analyses that indicated high degree of purity. Artificial proteoliposomes were prepared in the presence of 10 μg/ml RLIP76 purified from each source, and control proteoliposomes were prepared in the presence of 10 μg/ml crude membrane protein from each source. DOX binding to the filter membrane in the absence of proteoliposomes was determined from filtration of 14-¹⁴C-DOX in transport buffer, and subtracted from uptake observed in the presence of proteoliposomes to obtain vesicular uptake. Uptake was quantified by filtration 5 minutes after addition of buffer without or with 4 mM ATP to proteoliposomes. DOX-uptake in the absence or presence of ATP in RLIP76 and control liposomes is presented (FIG. 1 and TABLE 1). For FIG. 1, RLIP76 was purified by the DNP-SG affinity-sepharose purification procedure and reconstituted into artificial liposomes and ATP-dependent DOX-uptake was measured, such that the mean and standard deviations from three separate determinations are presented for recombinant RLIP76, the H182 and H1618 (SCLC) and H226 and H2347 (NSCLC) cell lines.

FIG. 1 shows that significantly greater DOX uptake was observed in the presence of ATP, only when recombinant or lung cancer cell line RLIP76 was present. ATP did not affect the DOX uptake in control liposomes. DOX transport rates calculated from uptake for SCLC cell lines (13.8 and 16.1 nmol/min/mg protein for H182 and H1618) were significantly lower that for RLIP76 from NSCLC cells (25.4 and 27.9 nmol/min/mg protein for H226 and H2347), or for recombinant RLIP76 (28.2 nmol/min/mg protein).

TABLE 1
14-¹⁴C-DOX uptake with or without ATP
in RLIP76 and control liposomes.

| Source | | | | |
|---|---|---|---|---|
| of | | | | RLIP76, |
| RLIP76 | Control, −ATP | Control, +ATP | RLIP76, −ATP | +ATP |
| H182 | 569 ± 99 | 656 ± 149 | 563 ± 128 | 2129 ± 191 |
| H1618 | 683 ± 84 | 544 ± 251 | 518 ± 183 | 2334 ± 383 |
| H226 | 673 ± 92 | 864 ± 275 | 830 ± 217 | 3356 ± 204 |
| H2347 | 479 ± 99 | 674 ± 127 | 685 ± 115 | 3215 ± 105 |
| Recom- | 729 ± 108 | 787 ± 142 | 739 ± 133 | 3434 ± 251 |
| binant | | | | |

Uptake of 14-¹⁴C-DOX by RLIP76- or control-proteoliposomes was measured 5 min after addition of proteoliposomes to transport buffer containing 3.6 μM 14-¹⁴C-DOX (8.4×10⁴ cpm/nmol). Aliquots of 40 μl transport buffer were filtered through the wells of the 96-well plate membrane filtration system. Background (6612±122 cpm, n=24) determined by filtering transport buffer containing 14-¹⁴C-DOX alone was subtracted from all values. Or control proteoliposomes prepared from each source were filtered. Values presented are average and standard deviations of background subtracted cpm values obtained from counting each membrane (n=3).

Comparison of DOX And VCR Transport Rates in Crude Membrane Vesicles from Cancerous Cells

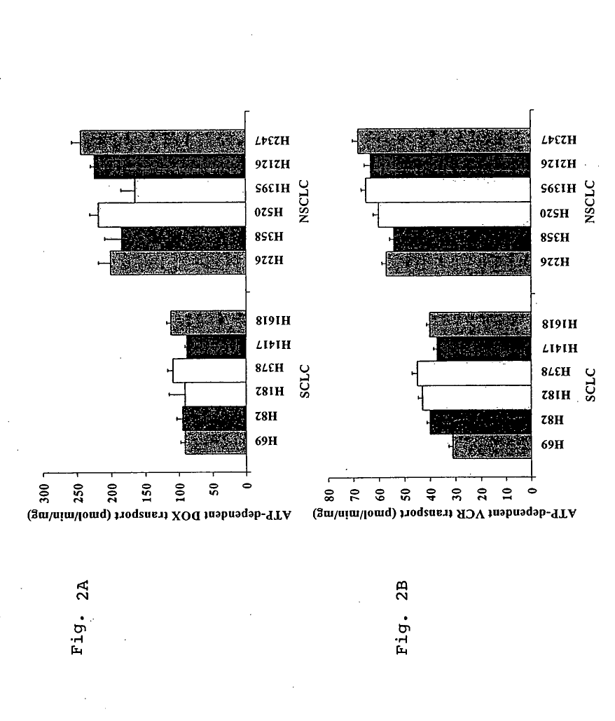

Crude membrane vesicles were prepared and enriched for inside-out vesicles as previously described (Awasthi, S. et al(2001). Biochemistry 39, 9327-9334; herein incorporated by reference) from six NSCLC and six SCLC cell lines. ATP-dependent transport of 14-¹⁴C-DOX and ³H-VCR was determined from subtraction of uptake in the absence of ATP from that observed in the presence of ATP. The rate of ATP-dependent DOX transport into crude membrane vesicles for NSCLC (206±22 pmol/min/mg vesicle protein, n=6) was about 2-fold higher than that for SCLC (98±9 pmol/min/mg vesicle protein, n=6) (FIG. 2, panel A). Likewise, VCR transport in NSCLC (61±5.2 pmol/min/mg, n=6) was higher than that observed in SCLC (39±4.9 pmol/min/mg) (FIG. 2, panel B). It should be noted that ATP-dependent DOX uptake rate in purified RLIP76-proteoliposomes (FIG. 1) was over 100 fold greater than the ATP-dependent DOX uptake rate in crude membrane vesicles (FIG. 2, panel A). This is consistent with over 100 fold purification of RLIP76 by the glutathione-conjugate affinity purification used here, and suggests that RLIP76 represents a significant fraction of total transporter activity in crude membrane vesicles.

Relative Contribution of RLIP76 Towards Transport

The relative contribution of RLIP76 transporters in the ATP-dependent transport of DOX in SCLC and NSCLC lines were quantified using an immunological approach. The inventors have previously shown that the polyclonal antibodies against RLIP76 inhibit DOX transport in membrane vesicles (Awasthi, S., et al. (1994) J. Clin. Invest. 93, 958-965; hererin incorporated by reference). Likewise, antibodies against MRP and Pgp also inhibit transport activity in crude membrane vesicles. Crude membrane vesicles were prepared from six SCLC and six NSCLC lines and separately coated with anti-RLIP76 IgG, anti-MRP1 IgG, or anti-MDR1 IgG were used to measure the ATP-dependent uptake of DOX.

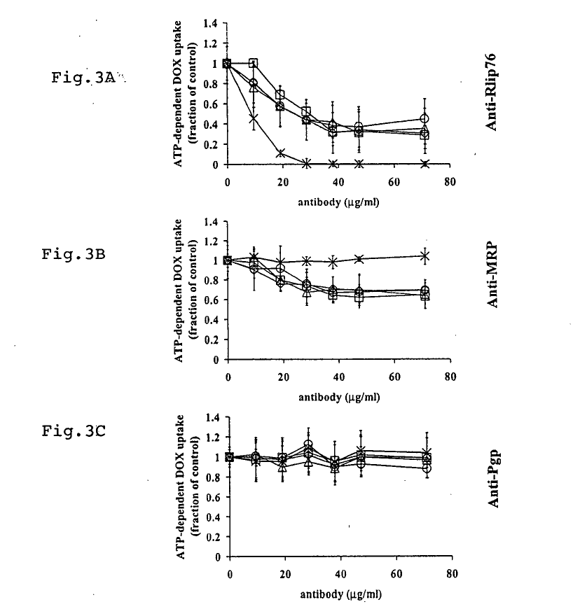

The specificity of each antibody used for its respective antigen was stringently established by Ouchterlony immunodiffusion assay which ruled out detectable cross-reactivity between these antibodies (data not shown). Optimal concentration of antibody to be used for specific inhibition of DOX transport was determined in titration studies where varying concentration of each antibody was used to coat the membrane vesicles. Results of these studies in crude membrane vesicles prepared from H182, H1618, H226 and H2347 cell lines are presented (FIG. 3). For FIG. 3, cholesterol liposomes were included. Membrane vesicles were incubated in the presence of varying concentration (10-100 μg antibody protein/ml transport reaction) for 30 minutes followed by determination of [14]-¹⁴C-DOX uptake by vesicles in the absence or presence of ATP as described and each point represents the mean of triplicate determinations with error bars representing standard deviations. Anti-RLIP76 antibodies completely abrogated transport seen in artificial proteoliposomes reconstituted with recombinant RLIP76, confirming the ability of these antibodies to inhibit RLIP76 mediated transport of DOX. Transport of DOX by recombinant RLIP76 (FIG. 3, panel A) was unaffected by anti-MRP or anti-Pgp antibodies, confirming the distinct immunological nature of RLIP76. In crude membrane vesicles from all four lung cancer cells, inhibition of DOX-transport by anti-RLIP76 IgG in a saturable manner with respect to antibody concentration was observed (FIG. 3, panel A). Unlike that observed with proteoliposomes reconstituted with purified recombinant-RLIP76, the anti-RLIP76 antibodies did not completely abrogate DOX transport in crude membrane vesicles from either SCLC or NSCLC cells. Maximal inhibition of DOX-transport in crude membrane vesicles from lung cancer cells by Anti-RLIP76 antibodies was remarkably similar for all the cell lines (65±7%). Anti-MRP antibodies also inhibited DOX transport in a saturable manner, but maximal inhibition was less (33±3%) as compared to that observed with anti-RLIP76 IgG (FIG. 3, panel B). Anti-Pgp antibodies had a small but detectable inhibitory effect on DOX transport (4±3%). These results also established that <50 μg/ml of each of the antibody quantitatively inhibited transport activity of their respective antigen present in the amount of vesicles used in these experiments.

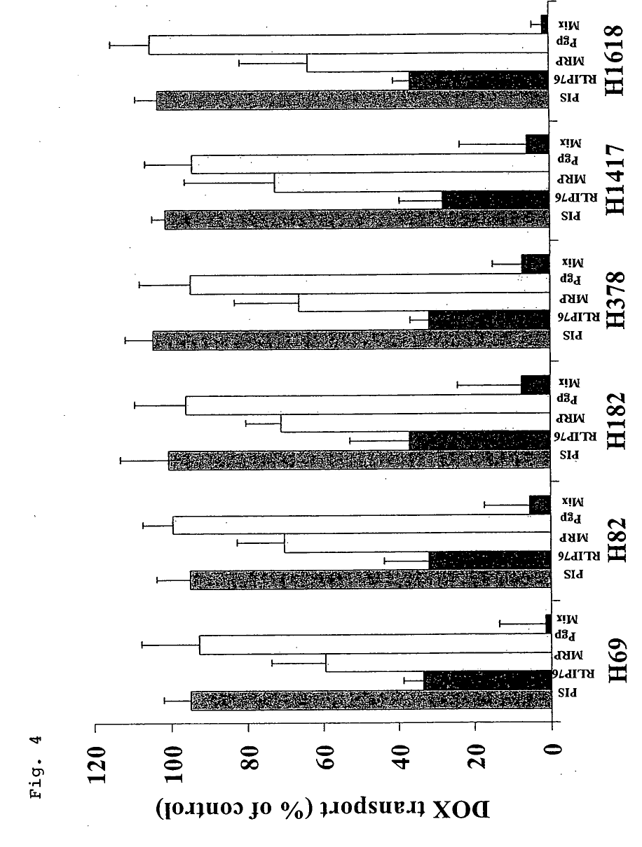

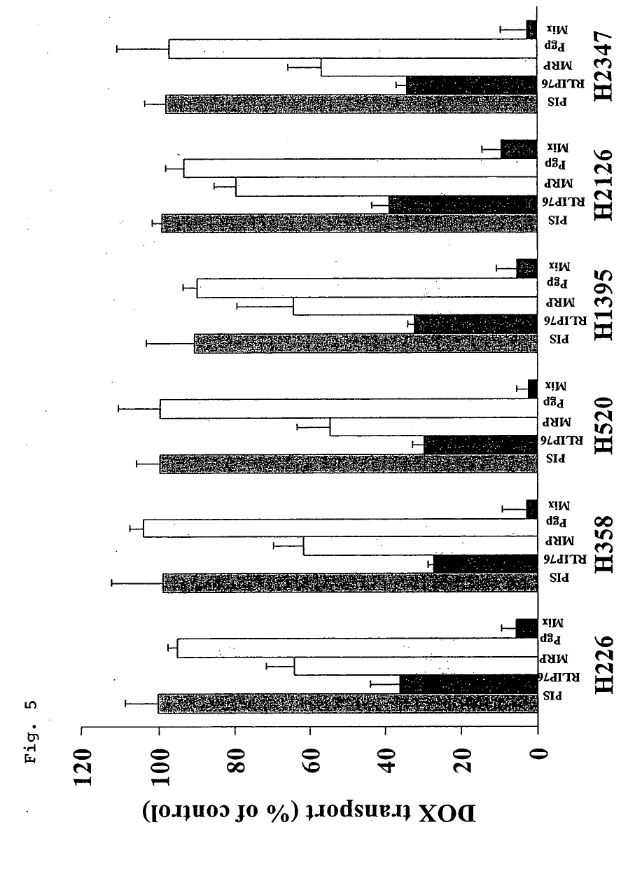

DOX transport was subsequently studied in crude membrane vesicles from all twelve cell lines, without or with coating in the presence of 50 μg/ml antibodies. Results of these studies normalized to control (without antibody) for SCLC cell lines (FIG. 4) and NSCLC cell lines (FIG. 5) are presented. For FIG. 4, crude membrane vesicles were prepared separately from each of six SCLC according to the method described above. Aliquots of IOV (30 μl) containing 20 μg protein were pre-incubated with 1 μg IgG from pre-immune serum or the specified antibodies for 30 min at 37° C. after which the ATP-dependent uptake of [14]-¹⁴C-DOX by IOV was measured in triplicate as previously described, sollowed ATP-dependent transport rates of [14]-¹⁴CDOX in the presence of pre-immune IgG (PIS) anti-RLIP76 IgG (RLIP76), anti-MRP IgG (MRP), anti-MDR1 IgG (Pgp), and the mixture containing 1 μg each of anti-RLIP76 IgG, anti-MRP IgG, and anti-MDR IgG (Mix) were normalized to the transport rate observed for each cell line in the absence of any antibody. Results of triplicate determinations with mean and standard deviation bars are presented for each of the six SCLC cell lines.

For FIG. 5, crude membrane vesicles were prepared from six NSCLC that were treated as described for FIG. 4, and ATP-dependent uptake of [14]-¹⁴C-DOX was measured as described previously. ATP-dependent transport rates of [14]-¹⁴C-DOX in the presence of pre-immune IgG (PIS) anti-RLIP76 IgG (RLIP76), anti-MRP IgG (MRP), anti-MDR1 IgG (Pgp), and the mixture containing 1 μg each of anti-RLIP76 IgG, anti-MRP IgG, and anti-MDR IgG (Mix) were normalized to the transport rate observed for each cell line in the absence of any antibody and results of triplicate determinations with mean and standard deviation bars are presented for each of the six NSCLC cell line.

FIGS. 4 and 5 show that about two-thirds of total DOX transport is inhibited in the crude membrane vesicles from all 12 cell lines coated with the anti-RLIP76 IgG (67±4%, n=12). Coating with anti-MRP1 IgG resulted in the inhibition of about one-third of total DOX transport (35±7%, n=12). Anti-MDR1 IgG did not significantly inhibit DOX transport activity (2±0.3%, n=12). In the vesicle coated with the mixture of the three antibodies almost complete abrogation (95±3%, n=12) of DOX transport was observed. In control vesicles coated with pre-immune IgG, the transport activity remained unaffected.

The present invention demonstrates that RLIP76, MRP, and Pgp together constitute nearly all ATP-dependent transport activity in these membranes. Furthermore, these results established that RLIP76 accounted for a major portion (>67%) of the ATP-dependent transport of DOX in cancer cells, such as those from the lungs. Together the above shows that RLIP76 mediates the ATP-dependent transport of chemotherapeutic agents such as DOX in cancerous growing cells (e.g., small cell, squamous cell, adenocarcinoma, bronchioalveolar carcinoma, and large cell carcinoma).

The transport activity of RLIP76 may be greater in some cancerous cells than others. For example, RLIP76 activity is greater in NSCLC as compared with SCLC. In both NSCLC and SCLC, it represents the major transporter of DOX. Transport activity of purified wild-type recombinant human RLIP76 was similar to that of RLIP76 purified from NSCLC cells. Despite equal amount of RLIP76 protein, the transport activity of RLIP76 from SCLC was about half that observed in RLIP76 from NSCLC and explains the underlying reasons for relative chemoresistance of NSCLC as compared with SCLC to this specific chemotherapeutic agent.

The ATPase activity of purified RLIP76 from 13 lung cancer cell lines was found to correlate with DOX-resistance in these SCLC and NSCLC cell lines. The activity of this protein is a strong determinant of DOX sensitivity. Since the ATPase activity of RLIP76 is required for DOX transport, the transport activity of RLIP76 in NSCLC appears greater than in SCLC. In addition, apparently homogenous RLIP76 from SCLC and NSCLC was reconstituted into artificial cholesterol:lecithin liposomes. The intra-/extra-vesicular volume ratio determined by radiolabeled inulin exclusion studies (18±2 μl intravesicular volume/ml reconstitution buffer) allows calculation of intravesicular DOX concentration from vesicular uptake.

The present invention also shows that the vesicular DOX concentration in the presence of both RLIP76 and ATP increased up to 10 fold above that observed in control liposomes without ATP, control liposomes with ATP, or RLIP76-liposomes without ATP (6.4±2 μM, n=45). The initial rate of transport could be calculated from uptake studies because this transport system shows linear increase in DOX accumulation with respect to time for up to 10 min. The initial rate of DOX-transport by RLIP76 from NSCLC cells was not significantly different from that observed with wild-type RLIP76 in present (data not shown), and about double that observed for RLIP76 from SCLC.

The present invention shows that the higher the ATPase activity of RLIP76 in a cell undergoing uncontrolled cell growth, the greater its transport activity.

Anti-RLIP causes a saturable inhibition of chemotherapeutic transport in crude membrane vesicles prepared from one or more cancerous cell lines. For example, measurements of DOX-transport inhibition by each individual antibody in all 12 cell lines demonstrated a remarkably consistent pattern which was also seen in the titration studies: about ⅔ of total DOX transport was inhibited by anti-RLIP76 and about ⅓ by anti-MRP. This doesn't rule out the involvement of other mechanisms in some cancerous cell lines (phosphorylation, ubiquitination, membrane permeability determinants, etc.), so that RLIP may be one of a combination of therapeutic targets in these cancerous cell lines (those that use more than one transport mechanism).

Additional Experimental Methods

Examples of reagents. Polyclonal rabbit-anti-RLIP76 IgG as well as pre-immune IgG were prepared and purified as is well known to one of ordinary skill in the art. For example, recombinant human RLIP76 may be expressed in E. Coli and purified by DNP-SG-Sepharose affinity purification as previously described. [See Awasthi, S., et al. (2001) Biochemistry 40, 4159-4168; herein incorporated by reference.] The antibody was injected (˜75 μg) into New Zealand White rabbit after obtaining pre-immune serum. After booster doses of 50 μg each at two-week intervals, post immune serum was obtained. The IgG fraction from pre- and post-immunized, heat-inactivated serum was purified by DE-52 anion exchange chromatography, followed by protein-A-Sepharose affinity chromatography. The purity of the antibody was checked by SDS-PAGE as well as Western-blotting against goat-anti-rabbit IgG. Aliquots of the antibody were stored at −86 degrees Centigrade and checked regularly by aerobic and anaerobic cultures for contamination. MRP1 (N-19) goat polyclonal IgG, human monoclonal trastuzumab, rituximab antibodies, human IgG, FITC-conjugated goat-anti-human and goat-anti-rabbit antibodies and [hu 14C]-DOX (specific activity 57 mCi/mmol) are commercially available.

Examples of cell lines and cultures. Human SCLC lines NCI-H82, NCI-H182, NCI-1417, NCI-1618, and NSCLC lines NCI-H1395, NCI-H2347, HCC44 (adenocarcinoma), NCI-H520, NCI-H226 (squamous cell carcinoma), NCI-H358 (bronchioalveolar) and NCI-H2126 (large cell) were studied. All cells were cultured at 37° C. in a humidified atmosphere of 5% CO₂ in RPMI-1640 medium supplemented with 10% (v/v) heat-inactivated FBS, 1% (v/v) P/S solution, 2 mM L-glutamine, 10 mM HEPES, 1 mM sodium pyruvate, 4.5 g/L glucose, and 1.5 g/L sodium bicarbonate.

Examples of purification of RLIP76 from NSCLC. DNP-SG affinity chromatography used for purification of RLIP76 from lung cancer cells uses methods known to one of ordinary skill in the art.

Examples of localization of RLIP76 on cell surface by flow-cytometry. Cell lines were harvested with cell dissociation media and were further dissociated into single-cell suspension by gently vortexing or rapid pipetting. The cells were then filtered through 70 μm Nylon cell strainers to get pure single cell populations, which were then seeded at a density of 1×10⁶ per ml in PBS containing 0.1% BSA and EDTA. Cells were treated with 50 μl of primary antibodies (stock 100 μg/ml) and incubated for 30 min at room temperature, washed twice with PBS buffer containing 0.1% BSA and EDTA, resuspended in 1 ml of PBS, and incubated for 30 min with 50 μl FITC-conjugated goat-anti-rabbit IgG (stock 20 μg/ml) as secondary antibodies. Cells were then washed again twice with PBS and re-suspended in 1% paraformaldehyde in PBS. Cells were stored at 4° C. in the dark until analyzed using FAC-Scan Cytometer (Becton-Dickinson). Controls included cells treated without primary antibody, with secondary antibody alone, and with rabbit pre-immune IgG.

Examples of immunohistochemical localization of RLIP76.

Immunohistochemical localization of RLIP76 was performed on NSCLC cells by the method readily apparent to one of ordinary skill in the art of immunochemistry. For examples, NSCLC cells were grown on cover slips. The cells were treated with either fixing medium containing 4% paraformaldehyde in PBS or PBS alone for 10 minutes at room temperature. Both fixed and unfixed cells were used for immunohistochemistry. Nonspecific antibody interactions were minimized by pre-treating cells with 10% goat serum in TBS for 30 minutes at room temperature. The cells were incubated with primary antibodies, anti-RLIP76 IgG or pre-immune IgG, for 2 hours at room temperature. After washing off the primary antibody with PBS (10 times, 3 minutes each), fluorescein isothiocyanate (FITC)-conjugated goat anti-rabbit immunoglobulin G (IgG) (1:50 dilution in PBS) as secondary antibodies were added and incubated for 1 hour at room temperature. The unbound secondary antibodies were removed by washing with PBS (10 times, 3 minutes each) and cover slips were air-dried and mounted on the slides with Vectashield mounting medium for fluorescence (Vector Laboratories, CA). Slides were photographed at 400× magnifications software and an inverted fluorescence microscope interfaced with a PC equipped with a CG7 frame grabber. Photographs were taken with 1-second integration.

Examples of drug sensitivity assay. The IC₅₀ of DOX was measured by MTT assay as described in part I of this communication. Eight replicate wells were used in each point in each of three separate measurement of IC₅₀. Measured absorbance values were directly linked with a spreadsheet for calculation of IC₅₀, defined as the antibodies or DOX concentration that reduced formazan formation by 50%.

Examples of Chou-Talalay median effect analysis of synergy. The Chou-Talalay method of determination of synergy is based on measurement of IC₅₀ values for each drug in alone and in combinations. The Calcusyn software package which utilizes an algorithm that takes into account the cytotoxicity as well as dose-effect curves for each chemotherapy drug and the antibodies was used. The method involves plotting dose-effect curves for each agent and combinations at various dilutions using the median effect equation as shown:

fₐ/fᵤ=(D/Dₘ)^{m} (1).

In this equation, Dₘ is the dose required for 50% effect, fₐ is the fraction affected by dose D and fᵤ is the fraction unaffected by dose D (I−fₐ). The exponent m is the coefficient of sigmoidicity (m=1, hyperbolic; m<1, negative sigmoid; m>1, sigmoid). This equation can be rearranged to yield:

D=Dm[fₐ/(1−fₐ)]^{1/m} (2).

The Dm and m values are determined from the median effect plot where x=log (D) and y=(fₐ/fᵤ). Linear regression was be used to determine m (slope) and log (Dₘ) (y-intercept). Analysis of synergy was performed by determining the combination index (CI) from the isobologram equation for two drugs (drug 1 and drug 2):

CI=(D)₁/(Dₓ)₁+(D)₂/(Dₓ)₂ (3).

CI<1,=1 and >1 correspond to synergy, additivity and antagonism.

Examples of determination of apoptosis by DNA laddering. Aliquots of 10×10⁶ cells/10 ml medium were grown in 75 mm dishes, treated separately overnight with medium alone or containing 50 μg/ml different IgG without or with DOX. The concentration of DOX was optimized to ensure visible DNA laddering in agarose gels. After 24 h incubation at 37 degrees Centirgrade, cells were collected, washed with PBS, counted by hemocytometer, and subjected to DNA extraction and agarose-gel electrophoresis according to the instructions in the DNeasy Tissue Kit (Quiagen, Inc.) or other available kits.

Inhibition of DOX efflux from lung cancer cells by various antibodies. The SCLC (H82) and NSCLC (H226 & H2126) cells lines were harvested washed with PBS, and aliquots containing 5×10⁶ cells (in triplicate) were inoculated into fresh medium. After overnight incubation, the cells were pelleted and re-suspended in 80 μl medium containing 5 μg respective IgG and incubated at 37° C. for 3 h. 14-[¹⁴C]-DOX (3.6 μM) or [³H]-4HNE (400 μM) was then added to the medium and incubated for additional 20 min at 37° C. Cells were centrifuged at 4° C. and medium was completely decanted and washed the cell pellet twice with PBS. Radioactivity was determined in the cell pellet. Counts were normalized to no-IgG control.

Membrane Association of RLIP76

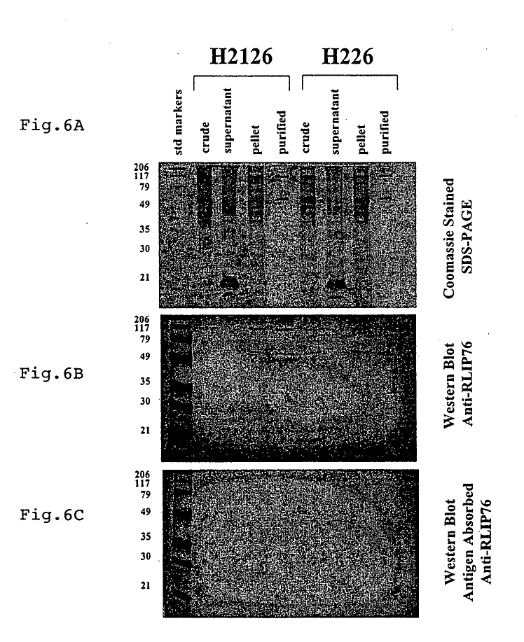

Subcellular localization of RLIP76 was investigated by fractionating whole cell homogenate from H2126 and H226 NSCLC cell lines by centrifugation at 28,000×g and analyzing these fractions by SDS-PAGE and Western-blot analyses against anti-RLIP76. The specificity of the anti-RLIP76 IgG for RLIP76, specifically the lack of cross-reactivity with MRP or Pgp was demonstrated by results of the Ouchterlony immunodiffusion analyses presented in part I of this communication. Purified RLIP76 fraction from both cells lines showed the characteristic 67 and 109 kDa bands in Coomassie-stained SDS-PAGE (FIG. 6, panel A). Absence of other detectable peptides in the purified RLIP76 fraction by SDS-PAGE, as well as the presence of the same pattern of bands in anti-RLIP76 Western-blot analysis (FIG. 6, panel B), indicated purity of the protein, which was also confirmed by amino acid composition analysis. For FIG. 6, polyclonal rabbit-anti-human RLIP76 IgG were prepared using crude membrane fraction, 28,000×g supernatant, resuspended pellet fraction, and DNP-SG affinity purified fraction from H2126 and H226 NSCLCL cell lines. The SDS-PAGE demonstrates purification of 109 kDa and 67 kDa peptide bands, wherein N-terminal sequence of the 109 kDa band revealed the N-terminal residues of RLIP76, and the 67 kDa band shown an internal sequence beginning at methionine 225. The middle panel shows that both bands were recognized by anti-RLIP76 IgG, in both crude and purified fractions and the bottom panel shows that the anti-RLIP76 IgG was completely absorbed by RLIP76-antigen immuno-affinity chromatography.

Western-blot analyses of the crude homogenate fraction and detergent-solubilized pellet fractions also showed only the two peptide bands characteristic for RLIP76, attesting to the specificity of anti-RLIP76 IgG. These bands were notably absent from the corresponding supernatant fractions in both cell lines, strongly indicating that RLIP76 was largely membrane associated in these NSCLC cells.

When anti-RLIP76 IgG was passed over an RLIP76-Sepharose antigen column, the unabsorbed fraction failed to recognize any bands in the crude or purified fractions (FIG. 6, panel C), indicating that anti-RLIP76 IgG antibodies were not contaminated with antibodies recognizing other proteins.

Cell Surface Localization of RLIP76

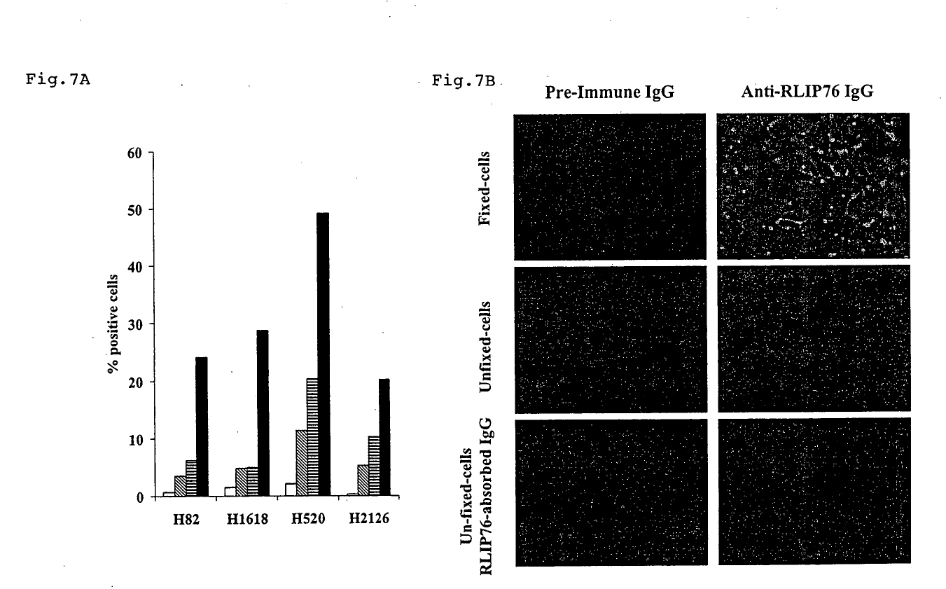

Because anti-RLIP76 IgG blocks transport of chemotherapeutic agents in crude vesicles and anti-RLIP76 antibodies can also inhibit the transport function of RLIP76 in intact cells, anti-RLIP76 antibodies must recognize some domain of RLIP76 at the external membrane surface. Flow-cytometric analysis was used to show this. In addition, it showed that anti-RLIP76 IgG was specifically bound to the outer surface of unfixed SCLC and NSCLC cells. In fact, a significant percentage of cells were detected by anti-RLIP IgG in both SCLC and NSCLC (FIG. 7, panel A), indicating that some domain(s) of RLIP76 recognized by these specific antibodies was present at the cell surface.

Cell surface localization of RLIP76 was also shown by comparing paraformaldehyde fixed vs. unfixed lung cancer cells by immunohistochemistry using pre-immune IgG, anti-RLIP76 IgG, as well as RLIP76-antigen-absorbed pre-immune and anti-RLIP76 IgG. For FIG. 7, the secondary antibody was an FITC-conjugated goat-anti-rabbit antibody. Slides were photographed at 400-× magnification using a Scion Image 1.62 C inverted fluorescence microscope interfaced with a PC equipped with a CG7 frame grabber. Photographs were taken with 1-second integration.

Representative results from the H358 NSCLC cell line are presented in FIG. 7. (FIG. 7, panel B). Whereas pre-immune IgG did show detectable cellular recognition, anti-RLIP76 IgG stained the paraformaldehyde fixed cells with the pattern of antigen recognition being consistent with the localization of RLIP76 on plasma membrane, nuclear envelope and nucleus (FIG. 7, panel B, top two photomicrographs).

If RLIP76 were associated only with the inner membrane leaflet, immunohistochemistry with unfixed cells should reveal no staining. To the contrary, anti-RLIP76 IgG stained the surface of unfixed H358 cells, while the pre-immune IgG did not bind significantly (FIG. 7, panel B, middle two photomicrographs). This antigen recognition at the surface of unfixed cells was abrogated by absorbing the anti-RLIP76 antibodies against RLIP76 antigen.

Taken together with examples showing that anti-RLIP76 IgG does not cross-react with other proteins in membrane fractions of cancer cells, these results show the existence of cell-surface domain(s) of RLIP76.

Anti-RLIP76 IgG Enhances Cytotoxicity of Chemotherapeutic Agents in Cancerous Growing Cells

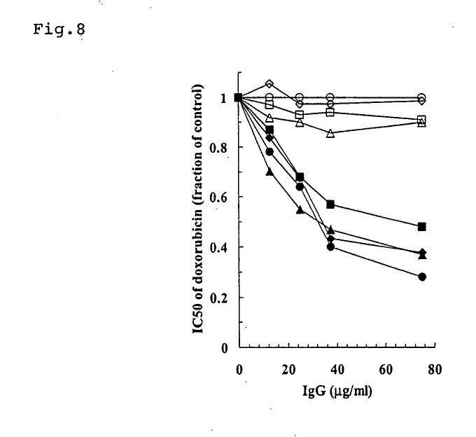

Anti-RLIP76 IgG inhibits the transport function of RLIP76 and it also enhances the cytotoxicity of chemotherapeutic agents. The effects of anti-RLIP76 IgG on DOX cytotoxicity towards SCLC and NSCLC lines is shown in FIG. 8. The IC₅₀ of DOX was determined for two SCLC (H82 and H182) and two NSCLC (H2126 and H2347) cells lines in the presence of several fixed concentrations of either pre-immune IgG or anti-RLIP76. Pre-immune IgG or anti-RLIP76 IgG was added to cells 16 hours before addition of DOX. MTT cytotoxicity assays were performed 96 hours after addition of DOX. IC₅₀ values at each concentration of pre-immune IgG or anti-RLIP76 were determined from plots of DOX concentration vs. OD₅₇₀. Results (FIG. 8) showed that anti-RLIP76 IgG caused a dose-dependent decrease in the IC₅₀ of DOX in all four cell lines. This effect was saturable, which was consistent with a specific antigen-antibody interaction.

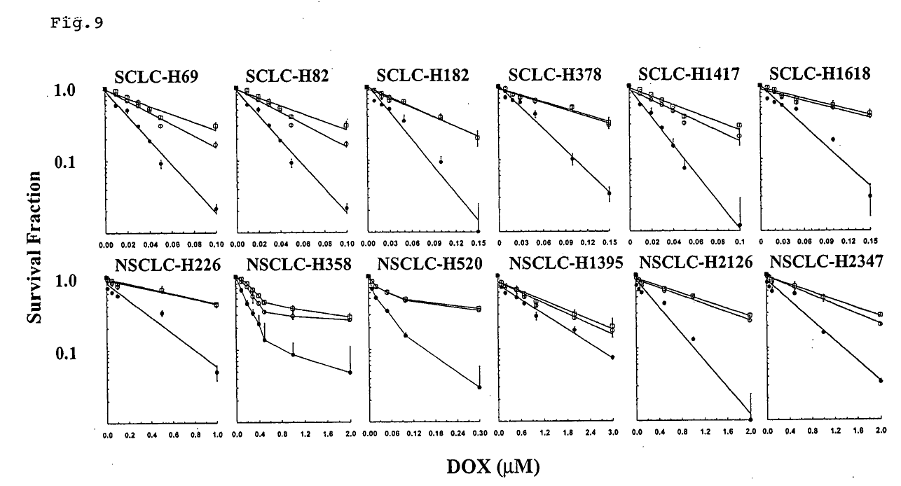

This effect of anti-RLIP76 IgG on DOX-cytotoxicity towards cells was further examined in cytotoxicity assays using one fixed concentration of antibody (50 μg/ml) with varying DOX concentrations in six SCLC and six NSCLC cell lines (FIG. 9). Whereas the DOX sensitivity of cells incubated with pre-immune IgG was unaffected, presence of anti-RLIP76 IgG significantly potentiated the cytotoxicity of DOX towards all the cell lines (p<0.001). As compared with the no antibody controls or pre-immune IgG, anti-RLIP76 IgG caused an order of magnitude greater cell kill at higher DOX concentrations for most of the SCLC and some of the NSCLC lines. The H358 and H520 cell lines differed from the others in having non-log-linear cell kill with DOX, perhaps due to heterogeneity in drug resistance mechanisms in the cell clones populating these cell lines. Overall, the sensitizing effect of anti-RLIP76 IgG to DOX was somewhat more prominent for SCLC as compared with NSCLC.

Anti-RLIP76 IgG Causes Apoptosis in Cancerous Growing Cells

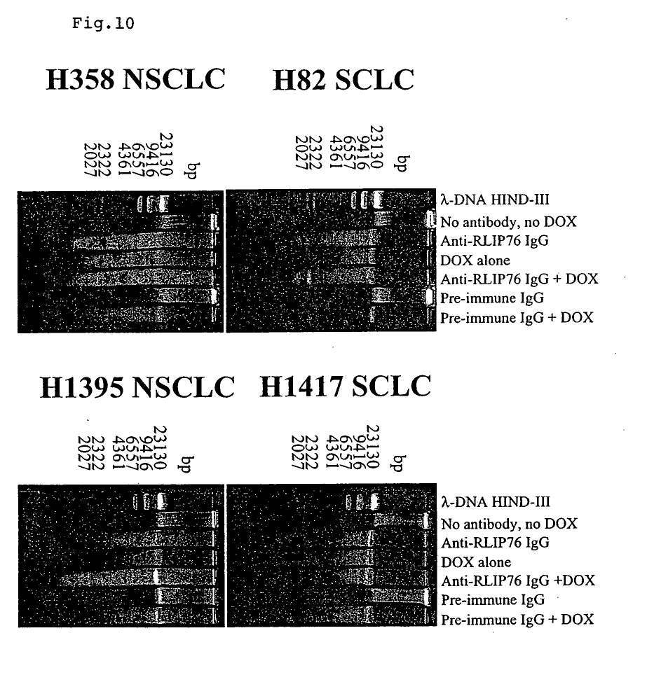

The mechanism of enhancement of DOX cytotoxicity by anti-RLIP76 antibodies is shown through the results of DNA-laddering, a hallmark of apoptosis. Cells were treated with DOX alone, pre-immune or anti-RLIP76 IgG alone, or in combination in a manner identical to that used for the MTT cytotoxicity assays, and DNA laddering was examined by agarose gel electrophoresis. Representative results from 2 SCLC (H82 and H1417) and two NSCLC (H358 and H1395) are presented (FIG. 10).

As expected, DOX alone caused the appearance of DNA laddering in all cell lines. Whereas pre-immune IgG alone caused no apoptosis, and did not appear to increase apoptosis caused by DOX, anti-RLIP76 increased DNA laddering in the presence of DOX. More interestingly, anti-RLIP76 alone also caused apoptosis in all the cell lines.

Synergistic Cytotoxicity of Anti-RLIP76 and Chemotherapeutic Agents

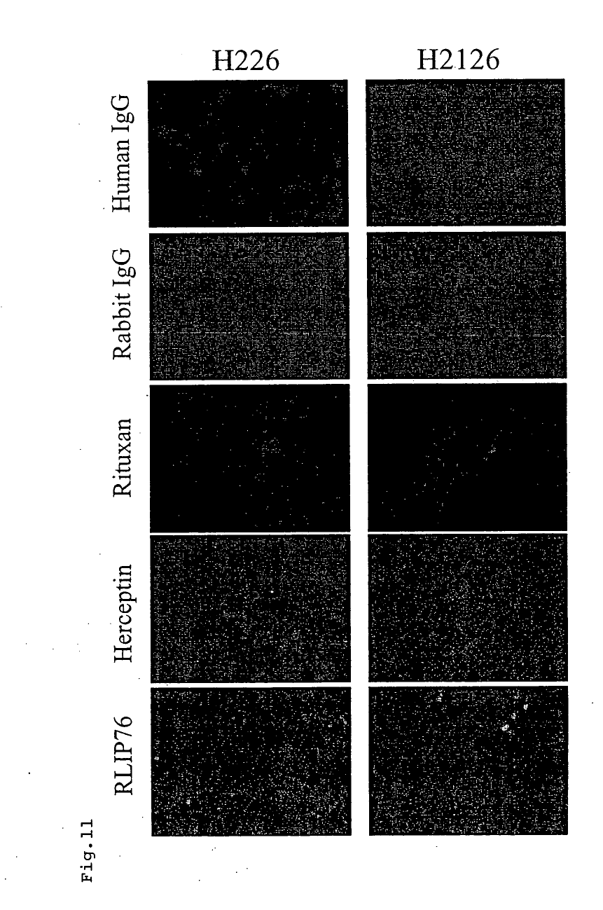

Because the anti-RLIP76 IgG itself appeared to exert cytotoxicity, the combined cytotoxicity of the two agents found to be due to synergistic cell kill. This was performed using the Chou-Talalay median effect analysis. Trastuzumab, an anti-Her-2/neu humanized monoclonal antibody was used. Expression of Her-2/neu has been observed in various cancer cells. Anti-RLIP76 and trastuzumab were separately tested at varying concentrations for cytotoxicity alone and in the presence of varying concentrations of DOX. Here, pre-immune IgG was used as a control for anti-RLIP76 and commercially obtained human IgG and rituximab (anti-CD20 antibody used in treatment of lymphoma) were used as negative controls. NSCLC cells were used because of their relatively higher resistance to DOX, and the ease of immunohistochemical studies with attached cells. The H226 and H2126 cell lines were chosen for study because of detectable expression of Her-2/neu. Results of immunohistochemical studies with these two cell lines using anti-RLIP76, trastuzumab, human IgG, rituximab, and rabbit pre-immune IgG presented (FIG. 11) demonstrated the detection of both Her-2/neu and RLIP76 in paraformaldehyde fixed cells.

For FIG. 11, cells were grown on cover-slips in 12 well plates by plating 1×10⁶ cells per well and after 24 h, the cells on the cover-slips were fixed by immersion in 4% paraformaldehyde in PBS for 10 minutes, followed by washing twice with PBS, such that non-specific binding was blocked by incubating cells with 10% goat-serum for 30 minutes at room temperature, followed by respective primary antibodies (400 μg/ml) were added and incubation was carried out for 2 hours then washing with PBS and secondary antibody (FITC-conjugated goat-anti-rabbit IgG for anti-RLIP, and FITC-conjugated goat anti-human IgG for trastuzumab and rituximab) was added (100 μg/ml), followed by 1 hour incubation at room temperature and excess secondary antibody was washed off with PBS, such that cover-slips were air-dried and placed face-down in mounting media on microscope slides and slides were photographed at 400× magnifications using Scion Image 1.62 c software using inverted fluorescence microscope interfaced with a PC equipped with a CG7 frame grabber and photographs were taken with 1-second integration. As expected pre-immune IgG, rituximab, or human IgG did not recognize surface proteins.

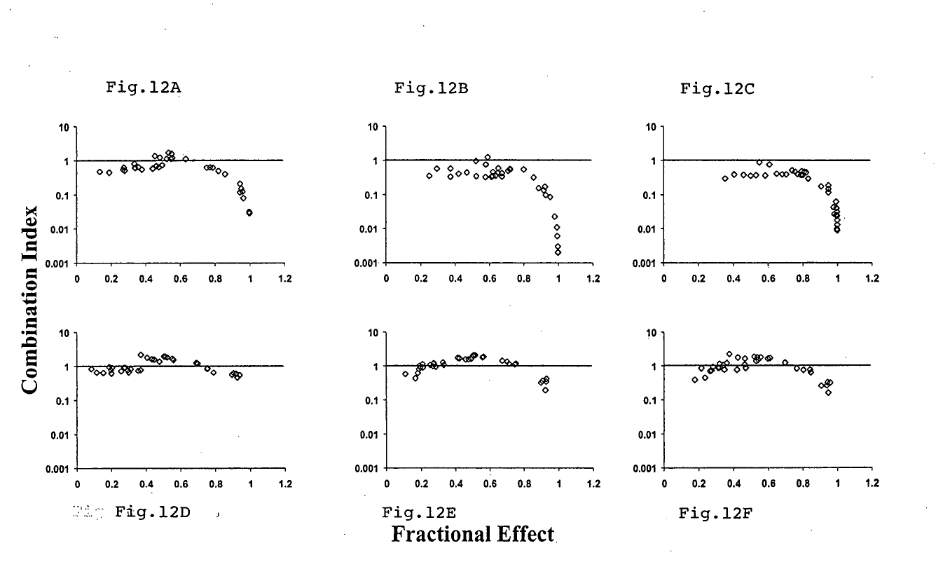

The cytotoxicity of DOX and each antibody towards H2126 was determined at varying concentration of each of the five antibodies and DOX separately and in combinations using an MTT assay. The Calcusyn software package was used to determine the fractional effect and combination index for each combination. DOX and trastuzumab were found to be significantly synergistic, with combination index near 0.01 at the highest concentration of DOX (FIG. 12, panel A). Anti-RLIP76 also potentiated DOX cytotoxicity in a synergistic fashion. The degree of synergy between anti-RLIP76 and DOX was found to be significantly greater (CI near 0.001) (FIG. 12, panel B) than that between DOX and trastuzumab. Presence of both anti-RLIP76 and trastuzumab did not further enhance synergy with DOX when the three agents were tested together (FIG. 12, panel C). As expected, neither human IgG (FIG. 12, panel D), rabbit pre-immune IgG (FIG. 12, panel E), nor the combination of both human IgG and rabbit IgG (FIG. 12, panel F) caused synergistic increase in DOX cytotoxicity.

For FIG. 12, the cytotoxicity of DOX alone and each antibody alone was determined in MTT assays with varying concentrations of respective agent and subsequently, the cytotoxicity of combinations of DOX and antibody was tested by varying the concentration of one and holding the concentration of the other one or two agents constant, such that eight replicate wells were used for each drug-antibody combination and data was analyzed using the Calcusyn software and the combination index was plotted against fractional effect derived from Calcusyn software analysis of data.

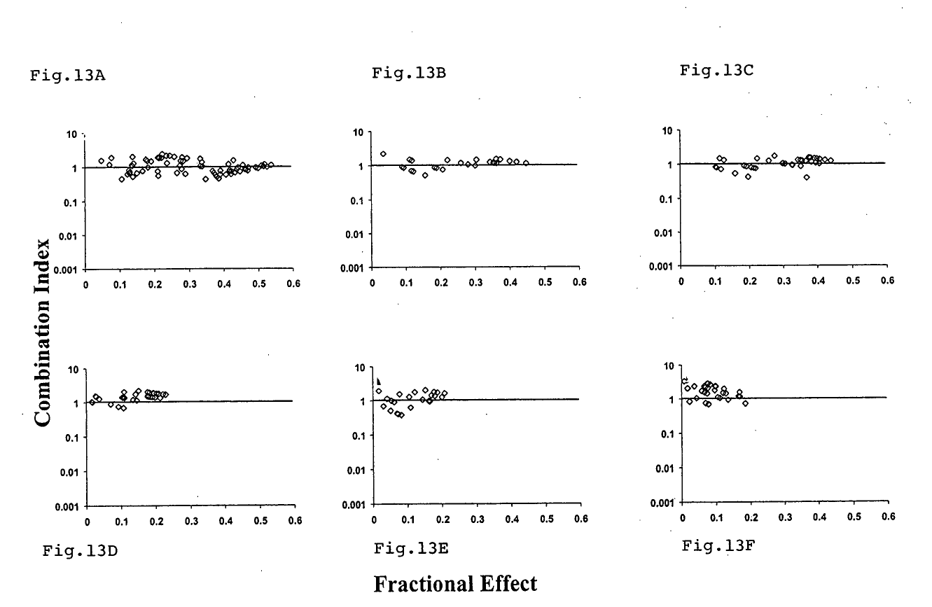

For cytotoxicity of combinations of antibodies (without DOX), no synergy was observed for the combinations of anti-RLIP76/human IgG (FIG. 13, panel B), anti-RLIP76/rituximab (FIG. 13, panel C), trastuzumab/rituximab (FIG. 13, panel D), trastuzumab/human IgG (FIG. 13, panel E), or rituximab/human IgG (FIG. 13, panel F). Only additive effect was observed with combination of anti-RLIP76 and trastuzumab (FIG. 13, panel A). For FIG. 13, the cytotoxicity of each antibody alone was determined in MTT assays with varying concentrations of respective agent and subsequently, the cytotoxicity of combinations of antibodies was tested by varying the concentration of one and holding the concentration of the other one or two agents constant, such that eight replicate wells were used for each antibody combination and data was analyzed using the Calcusyn software and combination index was plotted against fractional effect derived from Calcusyn software analysis of data.

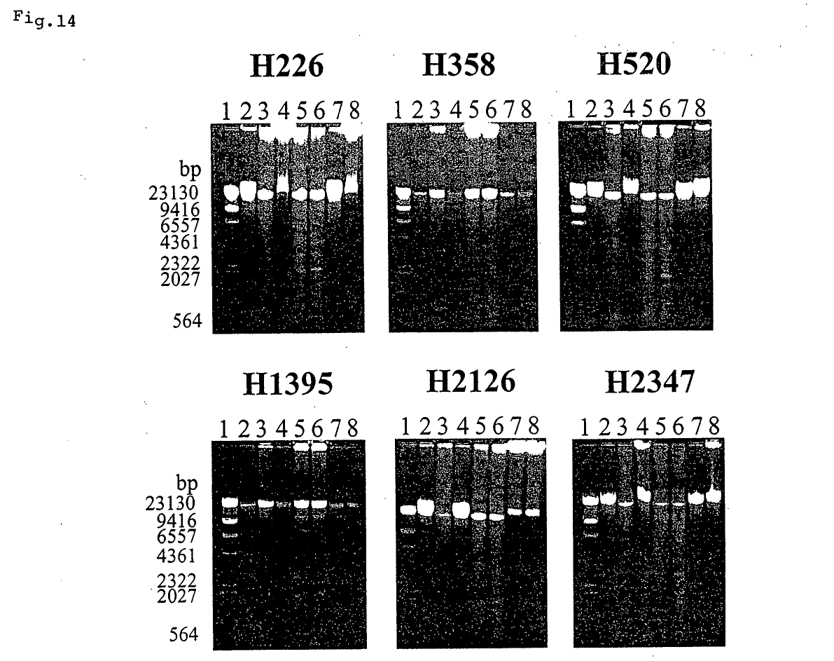

The anti-RLIP76/trastuzumab combination has an effect on apoptosis as evaluated by DNA laddering. Six NSCLC cell lines were tested for the DNA-laddering effect of these antibodies alone or in combination. Results show that anti-RLIP76 caused apoptosis in all six NSCLC cell lines (FIG. 14, lane 5). The effect of trastuzumab was less prominent and the degree of DNA laddering varied qualitatively between the cell lines. As predicted from the median effect analysis, trastuzumab did not markedly increase DNA-laddering caused by anti-RLIP76 alone. For FIG. 14, cells were diluted to a density of 1×10⁶ cells/ml in medium and 10 ml cells were added to petri dishes and after 24 hours, respective antibodies were added to a final concentration of 50 μg/ml, then after additional 24 hours incubation, medium was discarded, cells were washed with PBS and DNA was extracted using a Quiagen kit, and 5 μg DNA was applied to 1% agarose gel electrophoresis in 1×TAE buffer, such that in all panels and DNA was visualized by staining with ethidium bromide and gels photographed at 1× magnification.

Anti-RLIP76 Mediated Cytotoxicity

The present invention shows that anti-RLIP76 alone triggers apoptosis in cancerous growing cells and that this antibody synergistically enhanced cytotoxicity to chemotherapeutic agents. A physiologic function (i.e. glutathione-conjugate efflux) of RLIP76 exerts an anti-apoptotic effect, and the chemotherapeutic agent inhibits this physiologic function. For example, RLIP76 can regulate intracellular concentration of 4HNE (a pro-apoptotic byproduct of oxidative lipid metabolism) by mediating the efflux of the glutathione conjugate 4HNE-SG which is the chief metabolite of 4HNE (data not shown) and 4HNE-SG is an excellent substrate for transport by RLIP76. Furthermore, coating K562 cells with anti-RLIP76 antibodies results in increased intracellular 4HNE levels, as well as increased susceptibility to oxidants or heat-shock mediated apoptosis (data not shown).

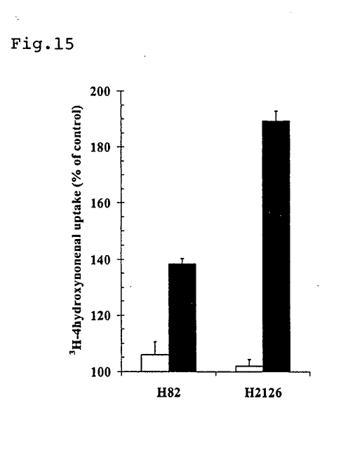

Intracellular 4HNE was also examined in other cancerous growing cells after exposing the cells (e.g., H82 SCLC and H2126 NSCLC) to exogenous radiolabeled 4HNE in the absence or presence of either pre-immune IgG or anti-RLIP76. Results of these studies showed that coating these cells with anti-RLIP76 results in a significant increase in cellular 4HNE levels, whereas the pre-immune IgG has little effect (FIG. 15). For FIG. 15, the effect of anti-RLIP76 IgG on intracellular accumulation of 4HNE are presented for the SCLC cell line, H82, and NSCLC cell line, H2126, such that for these studies, aliquots of 5×10⁶ cells washed with PBS were incubated with 50 μg/ml antibody (pre-immune IgG, open bar, or anti-RLIP76 IgG, filled bar) in medium for 3 hour at 37 degrees Centigrade. ³H-4HNE (400 μM, specific activity 7.7×10² cpm/nmol) was added and incubated at 37 degrees Centigrade for additional 20 minutes and cells were pelleted by centrifugation at 1,500×g and resuspended in 10 ml scintillation fluid for radioactivity counting. The uptake in presence of antibodies was normalized to uptake in absence of antibodies. Values, mean±SD, n=6 are presented as percent of the control.

Clearly RLIP76 transports several different chemotherapeutic agents (e.g., anthracyclines, DOX, 4HNE-SG). Anti-RLIP76 inhibits their efflux from the cancerous growing cells resulting in increased intracellular of the agent.

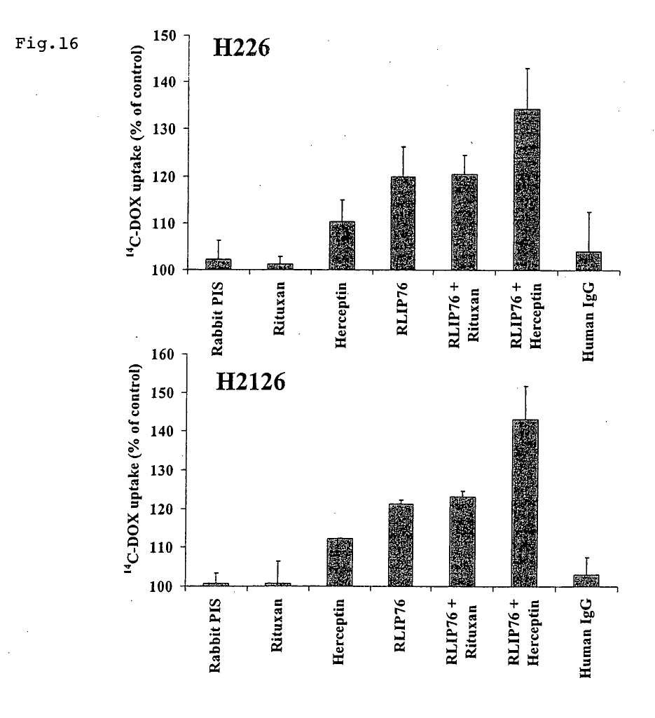

As an example, anti-RLIP76 IgG, pre-immune IgG, trastuzumab, rituximab, and human IgG effect differently the accumulation of a second chemotherapeutic agent in cancerous growing cells (e.g., H226 and H2126 NSCLC). For example, cells pre-incubated without or with each antibody for 3 hours, followed by addition of radiolabeled DOX were examined by terminating the assay via placing the cells on ice. Cellular DOX uptake was quantified after washing off extracellular medium. Neither rabbit pre-immune IgG, rituximab, or human IgG caused a significant change in cellular DOX accumulation (FIG. 16). Anti-RLIP76 as well as trastuzumab caused a significant increase in intracellular DOX. Whereas trastuzumab caused an additive increase in DOX accumulation when combined with anti-RLIP76 IgG, addition of rituximab had no effect.

The synergy between DOX anti-RLIP76 may from an inhibition of physiologic glutathione-conjugate efflux as well as inhibition of DOX efflux from cells. For FIG. 16, cells were harvested, washed with PBS and counted and aliquots containing 5×10⁶ cells were incubated in the absence or presence of each antibody (50 μg/ml) in medium at 37° C. waterbath, followed by centrifugation at 1500×g at 4 degrees Centigrade. The medium was then decanted and cells were washed with PBS twice before addition of counting cocktail to the cell pellet and uptake in the presence of antibodies was normalized to uptake in the absence of antibodies (mean and standard deviations from triplicate determinations in two separate experiments, n=6, are presented as percent of control).

As such, the present invention demonstrates that RLIP76 is the predominant transporter of chemotherapeutic agents in cancerous growing cells, that its transport and ATPase activity is greater in some cancerous growing cells (e.g., NSCLC) than other (e.g., SCLC), and that transport inhibition by anti-RLIP76 IgG augments the chemotherapeutic agents cytotoxicity through its increased accumulation in these cells. More importantly, these antibodies can trigger apoptosis in cancerous growing cells and can augment the apoptotic effects of a chemotherapeutic agent.

The present invention explains mechanisms through which cancerous growing cells evade the cytotoxic effects of anthracyclines and other chemotherapeutic agents as well as of mechanisms underlying the differential susceptibility of cancerous growing cells towards chemotherapy. Similar mechanisms underly radiation therapy, in that antibodies to RLIP augment the cytotoxicity of radiation therapy through an increased accumulation of its by-products. The present invention provides a novel mechanistic rationale for targeted modulation of chemotherapy and radiation therapy to improve the efficacy of chemotherapy in cancerous growing cells or by treating cancerous growing cells by inhibiting the transport and signaling functions of RLIP76.

Integral membrane association of RLIP76 was evident because the high-salt (150 mM NaCl) conditions used for its purification from human erythrocyte membrane vesicles would have removed RLIP76 if it were only loosely associated with membrane (data not shown). Recombinant RLIP76 expressed in E. coli was also found in the membrane fraction, extractable by non-ionic detergent (data not shown). The rathomolog of RLIP76, RalBP1, has also been clearly shown to be membrane associated in our studies (Swatko, M. E., et al. (2001) Chem. Biol. Interact. 133, 249-255; incorporated herein by reference). Requirement for detergent extraction has also been demonstrated for the N-terminal 49 kDa fragment of RLIP, which is highly sensitive to proteolysis and present largely in the membrane fraction (data not shown).

Proteolytic fragmentation of RLIP76, particularly in its N-terminal, may yield smaller peptides, which can rearrange to enable transport functionality. For example, the transport function of RLIP76 can be reconstituted when the C- and N-terminal fragments of RLIP76 are purified separately and reconstituted in artificial liposomes (data not shown). Therefore, peptides derived from RLIP76 are able to constitute a functional transporter in cell membranes.

Signaling of apoptosis by anti-RLIP76 IgG is explained by its inhibition of RLIP76 effector function as a transporter of the GSH-conjugate of 4HNE, another oxidative metabolite of arachidonic acid. 4HNE is generated in cells upon exposure to a number of stress situations or oxidant pro-apoptotic agents, including DOX and H₂O₂. 4HNE alone can trigger activation of the same elements of apoptotic pathways, including JNK, API and caspase, which are triggered in apoptosis caused by oxidants. In the presence of physiologic concentrations of GSH (1-2 mM) and GST, 4HNE is readily conjugated to 4HNE-S. RLIP76 is a significant contributor to the energy dependent efflux of 4HNE form cells (data not shown). In addition, apoptosis induced by inhibition of RLIP76 is accompanied, and perhaps preceded, by the activation of JNK, increased API-DNA binding, and caspase 3 activation (data not shown). Inhibition of 4HNE-SG transport by RLIP76 antibodies therefore increases the intracellular levels of 4HNE and consequently triggers apoptosis.

Because the effector function of RLIP76 as a transporter of 4HNE participates in regulating intracellular 4HNE concentration, its activity is also important in regulation of other cellular functions including cell growth and differentiation which is known to be affected by exposure to low (<2 μM) concentrations of 4HNE (data not shown). Thus, RLIP76 and other mechanisms that regulate 4HNE levels would function to minimize the duration of a ‘refractory state’ following a signal (EGF, TNF, etc.) which results in transient increases in cellular 4HNE. During this refractory state, 4HNE levels would be returned to below threshold levels, by the activity of GST, RLIP76, MRP, aldehyde reductases, and other catalytic activities (data not shown). As a result, cell growth may be signaled through mechanisms, which cause small increases in cellular 4HNE concentrations, whereas large or sudden increases in 4HNE as that which occur in the presence of potent oxidants could trigger apoptosis. The function of RLIP76 as an effector protein coupling ATP-hydrolysis with glutathione-conjugate efflux is consistent with an anti-apoptotic mechanism.

The GAP activity of RLIP76 functions to inhibit apoptosis by inhibiting signaling in ras-family pathways by inactivating ras-bound GTP through GTP hydrolysis (data now shown). The inventors have previously shown nucleotide binding domains in RLIP76 and GTP-hydrolyzing activity of RLIP76. Since RLIP76 hydrolyzes both free GTP and rho/rac-bound GTP, ras-family proteins appear as natural regulators of the nucleotidase activity of RLIP76. The ras proteins previously demonstrated to interact with RLIP76 include ra1, rac1 and cdc-42. Thus, the activity of RLIP76 may be controlled by proteins participating in cell growth, apoptosis, endocytosis, and movement. For example, GS-E are excellent natural substrates for transport by RLIP76 and shows that regulation of GS-E levels through the activity of RLIP76 is used by cells in a coordinated way in the signaling of these diverse cellular activities. Hence, ras-family proteins regulate the activity of each of RLIP76 effector functions by differentially targeting it to sites where effector organelles are present. Thus, the physiologic activities of nucleotide hydrolysis, and GS-E transport by RLIP76, and apoptosis triggered by inhibition of RLIP76 are certainly not inconsistent with the functions of RLIP76 predicted by other investigators or with the known functions of ras-linked signaling pathways.

Most importantly, the present invention illustrates that anti-RLIP76 IgG offers the advantage of a well defined biochemical mechanism of action as well as the lack of requirement of ADCC or CDC as, is often with other antibodies used for targeted therapy against one or more types of cancer.

In summary, the present studies demonstrate the presence of RLIP76 in cells with uncontrolled cell growth, and that the protein functions as a major transporter of drugs such as chemotherapeutic agents. The difference in activity of RLIP76 between different cancerous growing cells plays a significant role in the inherent differences in sensitivity to chemotherapeutic agents by different cancerous growing cell types. Therefore, inhibition of RLIP76 activity and its ability to synergistically enhance the cytotoxicity of a chemotherapeutic agent towards a cancerous growing cell can be readily examined by one of ordinary skill in the art of oncology. These antibodies are readily developed into humanized monoclonal antibodies that target one or more specific ligands in RLIP76.

In another form of the present invention is a method for synergistically enhancing the cytotoxicity of one or more chemotherapeutic agents and for enhancing apoptosis comprising the steps of contacting one or more cancerous growing cells with an antibody to an effective portion of RLIP76 and contacting one or more cancerous growing cells with one or more chemotherapeutic agents.

In still another form, the present invention is a pharmaceutical composition used to increases the accumulation of one or more chemotherapeutic agents inside a cancerous growing cell comprising one or more chemotherapeutic agents used in combination with an antibody to RLIP76, wherein the antibody comprises all or an effective portion thereof and when to bound a cancerous growing cell will promote the accumulation of one or more chemotherapeutic agents within the cell.

In yet another form, the present invention is a pharmaceutical composition used to enhance the exposure of one or more cancerous growing cells to radiation therapy comprising an effective dose of radiation therapy used in combination with an antibody to RLIP76, wherein the antibody comprises all or an effective portion thereof and when to bound a cancerous growing cell will promote the exposure of one or more cancerous growing cells to radiation therapy.

Another form of the present invention is a method of reducing the transport of 4HNE comprising the step of contacting one or more cancerous growing cells with an antibody to RLIP76, or a fragment of RLIP76, wherein the reduction in transport results in an intracellular accumulation of 4HNE. The antibody to RLIP76, or a fragment of RLIP76 is then administered to mammal. Routes of administration are any of those available for compositions comprising an antibody and are readily apparent to those of skill in the art of preparing pharmaceutical compositions.

The present invention is also a method of locating a cancerous growing cell comprising the step of contacting one or more cancerous growing cells with an antibody to RLIP76 that is connected to tracer molecule, wherein the tracer molecule is detected and identifies the location of the cancerous growing cell.

While specific alternatives to steps of the invention have been described herein, additional alternatives not specifically disclosed but known in the art are intended to fall within the scope of the invention. Thus, it is understood that other applications of the present invention will be apparent to those skilled in the art upon reading the described embodiment and after consideration of the appended claims and drawing.

What is claimed:

1. A method of treating one or more cells undergoing uncontrolled growth comprising the step of:
contacting one or more cells with an antibody to RLIP76.

2. The method of claim 1, wherein one or cells are selected from the group consisting of cancerous cells, NCI-H82, NCI-H182, NCI-1417, NCI-1618, NCI-H1395, NCI-H2347, HCC44 (adenocarcinoma), and NCI-H2126 (large cell), and combinations thereof.

3. The method of claim 1, wherein the antibody to RLIP76 inhibits the transport activity of RLIP76.

4. The method of claim 1, wherein the antibody is added in combination with a drug to prevent the drug from leaving the cell.

5. The method of claim 4, wherein the drug is used for chemotherapy.

6. The method of claim 1, wherein the antibody is selected from the group consisting of a mononclonal antibody, a polyclonal antibody, RLIP76 IgG raised against an effective portion of RLIP76.

7. The method of claim 4, wherein the drug is at least selected from the group consisting of doxorubicin, actinomycin, actinomycin D, altreatamine, asparaginase, bleomycin, busulphan, capecitabine, carboplatin, carmustine, chlorambucil, cisplatin, cyclophosphamide, cytarbine, dacarabazine, daunorubicin,, epirubicin, etoposide, fludarbine, fluorouracil, gemcitabine, herceptin, hydroxyurea, idarubicin, ifosfamide, irinotecan, lomustine, melphalan, mercaptopurine, methotrexate, mitomycin, mitozantrone, oxaliplatin, procarbazine, rituxan, steroids, streptozocin, taxol, taxotere, tamozolomide, thioguanine, thiotepa, tomudex, topotecan, treosulfan, uracil-tegufur, vinblastine, vincristine, vindesine, vinorelbine, and effective combinations and analogs thereof.

8. The method of claim 4, wherein addition of the drug to the antibody enhances the cytotoxicity of the drug.

9. The method of claim 4, wherein antibody promotes apoptosis of the cell.

10. The method of claim 1, wherein the antibody is added in combination with radiation therapy.

11. The method of claim 10, wherein the antibody in combination with radiation therapy enhances effectiveness of the radiation therapy.

12. A pharmaceutical composition for the treatment of one or more cells undergoing uncontrolled growth comprising:
an antibody to RLIP76, wherein the antibody comprises all or an effective portion thereof that effectively reduces the transport activity of RLIP76; and
a pharmaceutically effective carrier.

13. The pharmaceutical composition of claim 11, wherein the antibody is selected from the group consisting of a mononclonal antibody, a polyclonal antibody, RLIP76 IgG raised against an effective portion of RLIP76.

14. The pharmaceutical composition of claim 11, wherein the pharmaceutical composition is used in combination with a drug.

15. The pharmaceutical composition of claim 11, wherein addition of the drug to the pharmaceutical composition enhances the cytotoxicity of the drug.

16. The pharmaceutical composition of claim 11, wherein the pharmaceutical composition is used in combination with radiation therapy.

17. The pharmaceutical composition of claim 11, wherein radiation of the pharmaceutical composition enhances the effect of radiation treatment.

18. The pharmaceutical composition of claim 11, wherein the pharmaceutical composition promotes apoptosis of the cells.

19. A method of locating a cell undergoing uncontrolled growth comprising the step of:
contacting one or more cells with an antibody to RLIP76, wherein the antibody is connected to tracer molecule and the tracer molecule is capable of identifying the location of the cell.

20. The method of claim 17, wherein the antibody is administered to mammal.
